(19) United States
(12) Patent Application Publication
(10) Pub. No.: US 2010/0016374 A1
(43) Pub. Date: Jan. 21, 2010

(54) Compounds Which Inhibit the Glycine Transporter and Uses Thereof

(76) Inventors: Steven Coulton, Essex (GB); Martin Gilpin, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/295,628

(22) PCT Filed: Apr. 3, 2007
(86) PCT No.: PCT/EP2007/053275

§ 371 Date: Jul. 9, 2009

(30) Foreign Application Priority Data
Apr. 5, 2006 (GB) 0606880.3
Jun. 21, 2006 (GB) 0612340.0

(51) Int. Cl. A61K 31/166; C07D 211/06; C07D 207/06; C07C 233/78; C07D 401/06; A61K 31/44; A61K 31/40; A61K 31/4439

(52) U.S. Cl. 514/343; 546/234; 548/567; 564/185; 514/357; 514/428; 514/617

(57) ABSTRACT

Compounds of formula (I) and salts and solvates are provided:

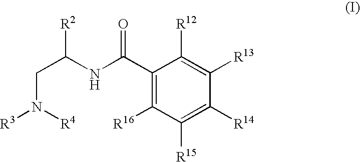

wherein R² is selected from phenyl substituted with n R¹ groups, and pyridyl substituted with n R¹ groups; n=0, 1 or 2; each R¹ is independently selected from the group consisting of halo, C₁₋₄alkyl, C₁₋₄alkoxy, haloC₁₋₄alkyl, haloC₁₋₄alkoxy and cyano; R³ is selected from hydrogen and C₁₋₂ alkyl; R⁴ is selected from the group consisting of ethyl, n-propyl, i-propyl, n-butyl, i-butyl and t-butyl; or R³ and R⁴ together with the nitrogen atom to which they are attached form a saturated 5- or 6-membered heterocyclic ring optionally substituted with one or more groups X; each X is independently selected from the group consisting of C₁₋₄alkyl, and haloC₁₋₄alkyl; R¹² is selected from the group consisting of hydrogen, fluoro, chloro, bromo, methyl and methylthio; R¹³ is selected from hydrogen, chloro and trifluoromethyl; R¹⁴ is selected from hydrogen, trifluoromethyl and chloro; R¹⁵ is selected from hydrogen, chloro and trifluoromethyl; R¹⁶ is selected from hydrogen, methyl, fluoro and chloro; R¹², R¹³, R¹⁴, R¹⁵ and R¹⁶ not all simultaneously being hydrogen.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-Ia, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science 262, 689-695 (1993); Lipton and Rosenberg, New Enql. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

A novel class of compounds which inhibit GlyT1 transporters have been found. The compounds are of potential use in the treatment of certain neurological and neuropsychiatric disorders, including schizophrenia.

Thus, in a first aspect, there is provided a compound of formula (I) or a salt or solvate thereof:

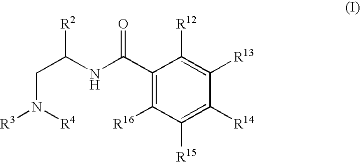

wherein
R² is selected from phenyl substituted with n R¹ groups, and pyridyl substituted with n R¹ groups;
n=0, 1 or 2;
each R¹ is independently selected from the group consisting of halo, C₁₋₄alkyl, C₁₋₄alkoxy, haloC₁₋₄alkyl, haloC₁₋₄alkoxy and cyano;
R³ is selected from hydrogen and C₁₋₂ alkyl;
R⁴ is selected from the group consisting of ethyl, n-propyl, i-propyl, n-butyl, i-butyl and t-butyl;
or R³ and R⁴ together with the nitrogen atom to which they are attached form a saturated 5- or 6-membered heterocyclic ring optionally substituted with one or more groups X;
each X is independently selected from the group consisting of C₁₋₄alkyl, and haloC₁₋₄alkyl;
R¹² is selected from the group consisting of hydrogen, fluoro, chloro, bromo, methyl and methylthio;
R¹³ is selected from hydrogen, chloro and trifluoromethyl;
R¹⁴ is selected from hydrogen, trifluoromethyl and chloro;
R¹⁵ is selected from hydrogen, chloro and trifluoromethyl;
R¹⁶ is selected from hydrogen, methyl, fluoro and chloro;
R¹², R¹³, R¹⁴, R¹⁵ and R¹⁶ not all simultaneously being hydrogen.

As used herein, the term “alkyl” refers to a straight or branched alkyl group in all isomeric forms. Examples of C₁₋₄alkyl include methyl, ethyl, propyl, isopropyl, butyl, isobutyl, sec-butyl and tert-butyl.

As used herein, the term “alkoxy” refers to the group —O-alkyl wherein alkyl is as defined above.

As used herein, the terms “halogen” and its abbreviations “hal” or “halo” refer to fluorine, chlorine, bromine, or iodine.

As used herein, the term “haloalkyl” refers to an alkyl group as defined above which is substituted with any number of fluorine, chlorine, bromine, or iodine atoms, including with mixtures of those atoms. A haloalkyl group may, for example contain 1, 2 or 3 halogen atoms. For example, a haloalkyl group may have all hydrogen atoms replaced with halogen atoms. Examples of haloalkyl groups include fluoromethyl, difluoromethyl and trifluoromethyl.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include water, ethanol and acetic acid. In one embodiment, the solvent used is water.

In one embodiment, R² is phenyl substituted with n R¹ groups.

In one embodiment, n is 0 or 1.

In one embodiment, each R¹ is independently selected from the group consisting of halo, C₁₋₂alkyl, C₁₋₂alkoxy, haloC₁₋₂alkyl, haloC₁₋₂alkoxy and cyano. In a further embodiment, each R¹ is independently selected from the group consisting of halo, methoxy and cyano. Examples of halo groups are fluoro and chloro, particularly fluoro.

In one embodiment, R³ is selected from hydrogen and methyl.

In one embodiment, R⁴ is selected from the group consisting of ethyl, n-propyl and i-propyl. In one embodiment, R⁴ is i-propyl.

In one embodiment, R³ and R⁴ together with the nitrogen atom to which they are attached form a saturated 5- or 6-membered heterocyclic ring optionally substituted with one or more groups X. In one embodiment, R³ and R⁴ together with the nitrogen atom to which they are attached form a saturated 5-membered carbocyclic ring optionally substituted with one or more groups X. In a further embodiment, R³ and R⁴ together with the nitrogen atom to which they are attached form a saturated 5-membered carbocyclic ring optionally substituted with one or two groups X.

In an embodiment, each X is independently selected from the group consisting of C₁₋₂alkyl, and haloC₁₋₂alkyl. For example, each X is independently selected from methyl and ethyl.

In one embodiment, R¹² is selected from hydrogen, fluoro, bromo, chloro and methyl.

In one embodiment, R¹² is selected from hydrogen and chloro. For example R¹² is chloro.

In one embodiment, R¹³ is selected from hydrogen and trifluoromethyl. For example R¹³ is trifluoromethyl.

In one embodiment, R¹⁴ is hydrogen.

In one embodiment, R¹⁵ is selected from hydrogen and trifluoromethyl.

In one embodiment, R¹⁶ is hydrogen.

In one embodiment, simultaneously: R¹² is chloro, R¹³ is trifluoromethyl, R¹⁴ is hydrogen, R¹⁵ is hydrogen, and R¹⁶ is hydrogen.

in one embodiment, the present invention provides a compound of formula (Ia) or a salt or solvate thereof:

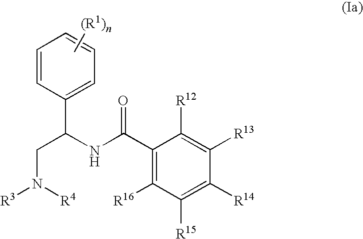

wherein
n=0, 1 or 2;
each R¹ is independently selected from the group consisting of halo, C₁₋₂alkyl, C₁₋₂alkoxy, haloC₁₋₂alkyl, haloC₁₋₂alkoxy and cyano;
R³ is selected from hydrogen and C₁₋₂ alkyl;
R⁴ is selected from the group consisting of ethyl, n-propyl, i-propyl, n-butyl, i-butyl and t-butyl;
or R³ and R⁴ together with the nitrogen atom to which they are attached form a saturated 5- or 6-membered heterocyclic ring optionally substituted with one or more groups X;
each X is independently selected from the group consisting of C₁₋₂alkyl, and haloC₁₋₂alkyl;
R¹² is selected from the group consisting of hydrogen, fluoro, chloro, methyl and methylthio;
R¹³ is selected from the group consisting of hydrogen, chloro and trifluoromethyl;
R¹⁴ is selected from hydrogen and chloro;
R¹⁵ is selected from the group consisting of hydrogen, chloro and trifluoromethyl;
R¹⁶ is selected from hydrogen and methyl;
R¹², R¹³, R¹⁴, R¹⁵ and R¹⁶ not all simultaneously being hydrogen.

In one embodiment, there is provided a compound of formula (Ib) or a salt or solvate thereof:

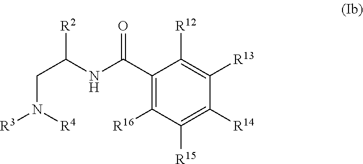

wherein
R² is selected from phenyl substituted with n R¹ groups, and pyridyl substituted with n R¹ groups;
n=0, 1 or 2;
each R¹ is independently selected from the group consisting of halo, C₁₋₄alkyl, C₁₋₄alkoxy, haloC₁₋₄alkyl, haloC₁₋₄alkoxy and cyano;
R³ is selected from hydrogen and C₁₋₂ alkyl;
R⁴ is selected from the group consisting of ethyl, n-propyl, i-propyl, n-butyl, i-butyl and t-butyl;
or R³ and R⁴ together with the nitrogen atom to which they are attached form a saturated 5- or 6-membered heterocyclic ring optionally substituted with one or more groups X;
each X is independently selected from the group consisting of C₁₋₄alkyl, and haloC₁₋₄alkyl;
R¹² is selected from the group consisting of hydrogen, fluoro, chloro and methyl;
R¹³ is selected from the group consisting of hydrogen, chloro and trifluoromethyl;
R¹⁴ is selected from hydrogen and chloro;
R¹⁵ is selected from the group consisting of hydrogen, chloro and trifluoromethyl;
R¹⁶ is selected from hydrogen and methyl;
R¹², R¹³, R¹⁴, R¹⁵ and R¹⁶ not all simultaneously being hydrogen.

It is to be understood that features of an embodiment of the invention described with reference to one parameter can be combined with the features of another embodiment. The disclosure herein thus includes the combination of the features of any one embodiment with the features of any other embodiment described. All embodiments and features of compounds of formula (I) apply to compounds of formula (Ia) and formula (Ib).

Examples of compounds of the invention include Examples 1 to 15 shown below, as well as salts and solvates thereof; or Examples 1 to 16 shown below, as well as salts and solvates thereof, ie:
2-Chloro-N-[(1S)-1-phenyl-2-(1-piperidinyl)ethyl]-3-(trifluoromethyl)benzamide
2-Chloro-N-[(1S)-1-phenyl-2-(1-pyrrolidi nyl)ethyl]-3-(trifluoromethyl)benzamide
2-Chloro-N-[(1S)-2-(2-methyl-1-pyrrolidinyl)-1-phenylethyl]-3-(trifluoromethyl)benzamide
2-Chloro-N-[(1S)-2-(2-ethyl-1-pyrrolidinyl)-1-phenylethyl]-3-(trifluoromethyl)benzamide
2-Chloro-N-[(1S)-2-(2,4-dimethyl-1-pyrrolidinyl)-1-phenylethyl]-3-(trifluoromethyl)benzamide
2-Chloro-N-{(1S)-2-[methyl (1-methylethyl)amino]-1-phenylethyl}-3-(trifluoromethyl)benzamide
2-Chloro-N-{(1S)-2-[(1-methylethyl)amino]-1-phenylethyl}-3-(trifluoromethyl)benzamide
2-chloro-N-[(1S)-1-(2-fluorophenyl)-2-(2-methyl-1-pyrrolidinyl)ethyl]-3-(trifluoromethyl)benzamide
2-chloro-N-[(1S)-1-(3-fluorophenyl)-2-(2-methyl-1-pyrrolidinyl)ethyl]-3-(trifluoromethyl)benzamide
2-chloro-N-[(1S)-1-(4-fluorophenyl)-2-(2-methyl-1-pyrrolidinyl)ethyl]-3-(trifluoromethyl)benzamide
2-chloro-N-[(1S)-1-(2-methoxyphenyl)-2-(2-methyl-1-pyrrolidinyl)ethyl]-3-(trifluoromethyl)benzamide
2-chloro-N-[(1S)-1-(3-methoxyphenyl)-2-(2-methyl-1-pyrrolidinyl)ethyl]-3-(trifluoromethyl)benzamide
2-chloro-N-[(1S)-1-(4-methoxyphenyl)-2-(2-methyl-1-pyrrolidinyl)ethyl]-3-(trifluoromethyl)benzamide
2-chloro-N-[(1S)-1-(3-cyanophenyl)-2-(2-methyl-1-pyrrolidinyl)ethyl]-3-(trifluoromethyl)benzamide
2-chloro-N-[(1S)-1-(4-cyanophenyl)-2-(2-methyl-1-pyrrolidinyl)ethyl]-3-(trifluoromethyl)benzamide
4-Chloro-2-methyl-6-(methylthio)-N-[(1S)-1-phenyl-2-(1-pyrrolidinyl)ethyl]benzamide
   or a salt or solvate thereof.

Further examples include:
2-chloro-N-{(1S)-2-[(2R,5S)-2,5-dimethyl-1-pyrrolidinyl]-1-phenylethyl}-3-(trifluoromethyl)benzamide
2-chloro-N-{(1S)-2-[(2R,6S)-2,6-dimethyl-1-piperidinyl]-1-phenylethyl}-3-(trifluoromethyl)benzamide
2-chloro-N-[(1R)-1-phenyl-2-(1-pyrrolidinyl)ethyl]-3-(trifluoromethyl)benzamide
2-chloro-N-[(1R)-2-(2-methyl-1-pyrrolidi nyl)-1-phenylethyl]-3-(trifluoromethyl)benzamide
2-fluoro-N-[(1S)-2-(2-methyl-1-pyrrolidinyl)-1-phenylethyl]-3-(trifluoromethyl)benzamide
3-chloro-2-fluoro-N-[(1S)-2-(2-methyl-1-pyrrolidinyl)-1-phenylethyl]-5-(trifluoromethyl)benzamide
2-chloro-N-[(1S)-2-(2-methyl-1-pyrrolidinyl)-1-phenylethyl]-5-(trifluoromethyl)benzamide
2-chloro-N-[(1S)-2-(2,2-dimethyl-1-pyrrolidinyl)-1-phenylethyl]-3-(trifluoromethyl)benzamide
2-chloro-N-{(1S)-2-[2-(1-methylethyl)-1-pyrrolidinyl]-1-phenylethyl}-3-(trifluoromethyl)benzamide
2,3-dichloro-N-[(1S)-2-(2-methyl-1-pyrrolidinyl)-1-phenylethyl]benzamide
2,3,5-trichloro-N-[(1S)-2-(2-methyl-1-pyrrolidinyl)-1-phenylethyl]benzamide
2,4-dichloro-N-[(1S)-2-(2-methyl-1-pyrrolidinyl)-1-phenylethyl]benzamide
2-chloro-N-[2-(2-methyl-1-pyrrolidinyl)-1-(2-pyridinyl)ethyl]-3-(trifluoromethyl)benzamide
2-chloro-N-[2-(2-methyl-1-pyrrolidinyl)-1-(3-pyridinyl)ethyl]-3-(trifluoromethyl)benzamide
2,6-dimethyl-N-[(1S)-2-(2-methyl-1-pyrrolidinyl)-1-phenylethyl]benzamide
2-bromo-6-methyl-N-[1-phenyl-2-(1-pyrrolidi nyl)ethyl]benzamide
   or a salt or solvate thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

The compounds described herein exist in stereoisomeric forms as they contain an asymmetric carbon atom:

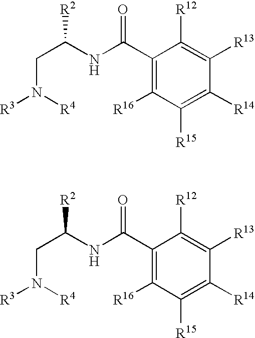

The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

In one embodiment, an optically pure enantiomer is provided. The term “optically pure enantiomer” means that the compound contains greater than about 90% of the desired isomer by weight, such as greater than about 95% of the desired isomer by weight, or such as greater than about 99% of the desired isomer by weight, said weight percent based upon the total weight of the isomer(s) of the compound. In some cases, one enantiomer of a particular structure may have a significantly higher activity than the other enantiomer of the same structure. Chirally pure, or chirally enriched compounds may be prepared by chirally selective synthesis or by separation of enantiomers. The separation of enantiomers may be carried out on the final product or alternatively on a suitable intermediate.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown in the following schemes. The starting materials and reagents are known to the skilled person in the art and/or can be prepared using methods known in the art.

Compounds of formula (I) can be synthesised by known methods; for example by, but not limited to, the synthetic route outlined in Scheme 1 below.

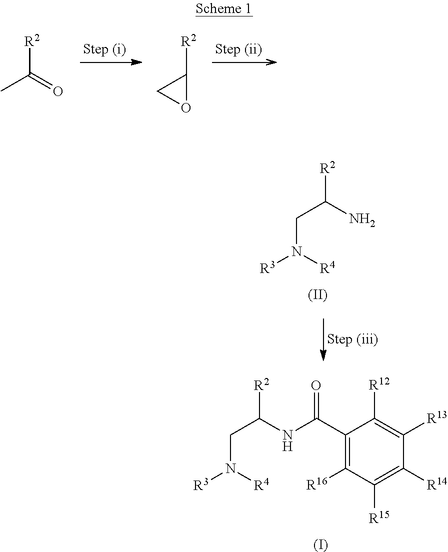

wherein R², R³, R⁴, R¹², R¹³, R¹⁴, R¹⁵, and R¹⁶ are as defined for the compound of formula (I).

For compounds in which R² is phenyl and n=0 (i.e. R² is unsubstituted phenyl), the epoxide compound preparable by step (i) is commercially available in racemic or enantiomerically pure form (for example in the (S) isomer form). When R² is a group such that the epoxide compound is not commercially available, the oxirane/epoxide may be prepared by a reaction shown in step (i) from a suitable ketone. That step may be carried out in an enantiomerically selective manner.

Step (i) is carried out for example by successive reaction of a ketone with suitable reagent(s) for introducing a leaving group in the position α to the carbonyl group, for example using [hydroxy(tosyloxy)iodo]benzene (J. S. Lodaya and G. F. Koser, J. Org. Chem., 1988, 53, 210), in a suitable solvent such as acetonitirile, or using bromine in acetic acid or methanol; followed by reduction of the carbonyl group with a suitable reducing agent, for example a borane reducing agent, such as borane-N-ethyl-N-isopropylaniline complex optionally in the presence of a suitable enantioselective chiral catalyst, for example a chiral oxazaborolidine, such as (S)-(−)-2-methyl-CBS-oxazaborolidine, in a suitable solvent such as tetrahydrofuran (B. T. Cho, W. K. Yang and O. K. Choi, JCS Perkin 1, 2001, 1204), followed by cyclisation to the oxirane by reaction with a base, for example an alkali metal hydroxide, such as sodium hydroxide, with a suitable solvent such as diethyl ether; or potassium carbonate in acetone.

Step (ii) can be achieved by successive reaction of the epoxide/oxirane with a compound of formula (IV):

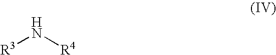

wherein R³ and R⁴ are as defined in formula (I), in a suitable solvent for example ethanol, followed by conversion of the alcohol to a suitable leaving group, for example by reaction with a reagent such as methanesulfonyl chloride, in the presence of a suitable base, such as triethylamine, in a suitable solvent, for example dichloromethane, followed by conversion to an amine, for example by reaction with aqueous ammonia, in a suitable solvent system. The solvent system may be monophasic, such as tetrahydrofuran; the solvent system may be biphasic, such as as mixture of diethylether and aqueous ammonia.

Acylation step (iii) can be achieved by reaction of a compound of formula (II) with a compound of formula (III):

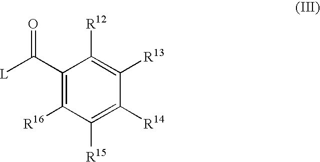

wherein R¹², R¹³, R¹⁴, R¹⁵, and R¹⁶ are as defined in formula (I) and L represents a suitable leaving group. Examples of leaving groups include halogen, hydroxy, OC(═O)alkyl, OC(═O)O-alkyl and OSO₂Me. L may be halogen and acylation in step (iii) may be carried out in an inert solvent such as dichloromethane, in the presence of a base such as triethylamine. When L represents hydroxy, the reaction preferably takes place in an inert solvent such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), polymer-supported EDC, polymer-supported DCC, O-(7-azabenzotriazol-1-yl)-1,1,3,3-tetramethyluronium hexafluoro phosphate (HATU), and optionally in the presence of 1-hydroxybenzotriazole hydrate (HOBT).

Within the scheme there is scope to convert a group R³ into another group R³ and similarly for a group R⁴.

Compounds of formula (I) can also be synthesised by the synthetic route outlined in Scheme 2 below.

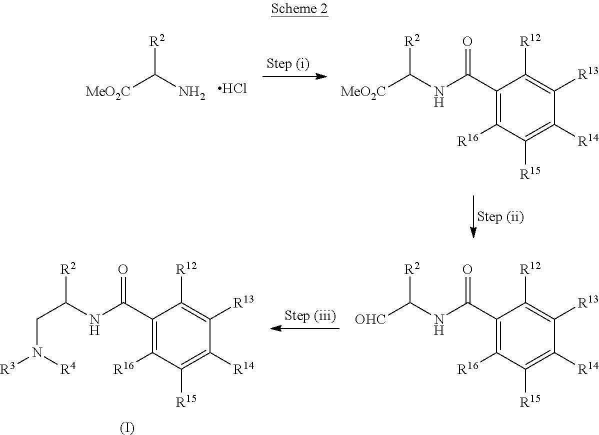

wherein R², R³, R⁴, R¹², R¹³, R¹⁴, R¹⁵, and R¹⁶ are as defined for the compound of formula (I). The route set out in Scheme 2 is particularly suitable for compounds in which R³ is H.

Acylation step (i) can be achieved by reaction with a compound of formula (III):

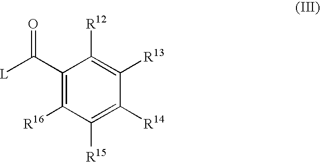

wherein R¹², R¹³, R¹⁴, R¹⁵, and R¹⁶ are as defined in formula (I) and L represents a suitable leaving group. Examples of leaving groups include halogen, hydroxy, OC(═O)alkyl, OC(═O)O-alkyl and OSO₂Me. L may be halogen and acylation in step (iii) may be carried out in an inert solvent such as dichloromethane, in the presence of a base such as triethylamine. When L represents hydroxy, the reaction preferably takes place in an inert solvent such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), polymer-supported EDC, polymer-supported DCC, O-(7-azabenzotriazol-1-yl)-1,1,3,3-tetramethyluronium hexafluoro phosphate (HATU) and optionally in the presence or 1-hydroxybenzotriazole hydrate (HOBT).

Step (ii) is carried out for example by reaction with a suitable reducing agent, for example diisobutylaluminium hydride, in a suitable inert solvent such as dichloromethane or tetrahydrofuran.

Step (iii) can be achieved by reaction with a compound of formula (IV):

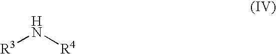

wherein R³ and R⁴ are as defined in formula (I), in the presence of a suitable reducing agent, for example sodium triacetoxyborohydride or sodium cyanoborohydride, and a suitable acid, such as acetic acid, in a suitable solvent for example dichloromethane.

Accordingly, in a second aspect, the present invention provides a method of preparing a compound of formula (I), comprising the step of:

reacting a compound of formula (II):

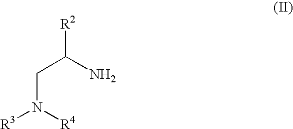

wherein R², R³ and R⁴ are as defined in formula (I), with a compound of formula (III):

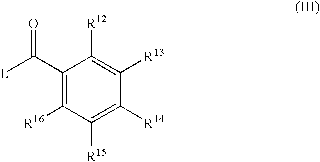

wherein R¹², R¹³, R¹⁴, R¹⁵, and R¹⁶ are as defined in formula (I) and L represents a suitable leaving group;
and thereafter optionally:
removing any protecting groups and/or
converting a compound of formula (I) into another compound of formula (I) and/or
forming a salt or solvate.

Suitable leaving groups L include halogen, hydroxy, OC(═O)alkyl, OC(═O)O-alkyl and OSO₂Me.

Compounds of formula (I) can be converted into further compounds of formula (I) using standard techniques. For example, and by way of illustration rather than limitation, possible conversion reactions include acylation with an appropriate acylating agent such as acetyl chloride, alkylation using an appropriate alkylating reagent such as methyl iodide, and sulfonylation using a sulfonylating agent such as methanesulfonic anhydride and N-alkylation by reductive amination using a ketone or an aldehyde in the presence of a reducing agent such as sodiumtriacetoxyborohydride.

Pharmaceutically acceptable salts may be prepared conventionally by reaction with the appropriate acid or acid derivative.

In a further aspect, the present invention provides a compound of formula (II):

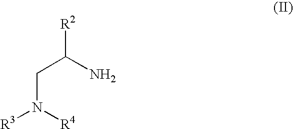

wherein R², R³ and R⁴ are as defined in formula (I).

Compounds of formula (II) are useful as intermediates in the synthesis of compounds of the invention.

The compounds of the present invention inhibit the GlyT1 transporter. The compounds may selectively inhibit the GlyT1 transporter over the GlyT2 transporter. Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by any of the following assays:

1) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 1.32×10⁶ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension (1.32×10⁶) and 5 uL of the cell/bead suspension transferred to each well of a 384-well white solid bottom plate (3300 cells/well) containing 100 nL of test compounds. Substrate (5 uL) was added to each well [1:100 dilution of [³H]-glycine stock in assay buffer containing 2.5 uM glycine). Final DMSO concentration was 1% v/v. Data was collected using a Perkin Elmer Viewlux. pIC₅₀ values were determined using ActivityBase.

2) HEK293 cells expressing the Glycine (Type 1) transporter are grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks are harvested and resuspended at 4×10⁵ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker™ SPA beads (12.5 mg/ml suspended in assay buffer) is added to the cell suspension. Compounds are prepared as 10 mM stocks in DMSO. 2.5 fold serial dilutions of the compounds are made in DMSO from a top conc of 2.5 mM. 100 nL of compound at each concentration is added to the assay plate (384-well white solid bottom plate) using the hummingbird dispenser. 5 uL of the cell/bead mix is then added on top of the compound using a multidrop dispenser. Substrate (5 uL) is then added to each well (1:100 dilution of H3-glycine in assay buffer containing 2.5 uM glycine) Data is collected using a PerkinElmer Viewlux as 5 minute exposures. pIC50 data values are determined using Activity Base.

3) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37 C and 5% CO2. Cells grown to 70-80% confluency in T175 flasks were harvested and frozen. For the assay, cells were defrosted and resuspended at 1.32×106 cells/mL in assay buffer [140 mM NaCl, 5.4 mM KCl, 1.8 mM CaCl2, 0.8 mM MgSO4, 20 mM HEPES, 5 mM glucose and 5 mM alanine, pH 7.4]. Compounds were serially diluted 4-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension (1.32×106) and 5 uL of the cell/bead suspension transferred to each well of a LV384-well white solid bottom plate (3300 cells/well) containing 100 nL of test compounds. Substrate (5 uL) was added to each well [1:100 dilution of [3H]-glycine stock in assay buffer containing 2.5 uM glycine). Final DMSO concentration was 1% v/v. Data was collected using a Perkin Elmer Viewlux. pIC50 values were determined using ActivityBase.

Compounds may be assayed in their free base form or in the form of a salt, for example the hydrochloride salt or the formate salt. The assays described above are generally considered to provide data that is correct to ±3 standard deviations=±0.5.

Compounds having a pIC₅₀ at the GlyT1 transporter of greater than or equal to 5.0 are considered to be active at the GlyT1 transporter. The example compounds below were found to have a pIC₅₀ at the GlyT1 transporter of greater than or equal to 5.0, in one or more of the above assays.

In a further aspect of the invention, there is provided a compound of formula (I) or a salt or solvate thereof for use in therapy.

In another aspect of the invention, there is provided a compound of formula (I) as hereinbefore described or a salt or solvate thereof, for use in the treatment of a disorder mediated by GlyT1.

In order to use a compound of the present invention as a medicament, it will normally be formulated into a pharmaceutical composition in accordance with standard pharmaceutical practice. The present invention also provides a pharmaceutical composition, which comprises a compound of formula (I) or a salt or solvate thereof, and a carrier, diluent or excipient.

In a further aspect, the present invention provides a process for preparing a pharmaceutical composition, the process comprising mixing a compound of formula (I) or a salt or solvate thereof and a carrier, diluent or excipient.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) may be also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-induced Mood Disorder (including the subtypes With Depressive Features, With Manic Features and With Mixed Features) and Mood Disorder Not Otherwise Specified (296.90).

The compounds of formula (I) may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder and Anxiety Disorder Not Otherwise Specified (300.00).

The compounds of formula (I) may also be of use in the treatment of substance-related disorders including Substance Use Disorders such as Substance Dependence and Substance Abuse; Substance-induced Disorders such as Substance Intoxication, Substance Withdrawal, Substance-induced Delirium, Substance-induced Persisting Dementia, Substance-induced Persisting Amnestic Disorder, Substance-induced Psychotic Disorder, Substance-induced Mood Disorder, Substance-induced Anxiety Disorder, Substance-induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-induced Persisting Amnestic Disorder, Alcohol-induced Psychotic Disorder, Alcohol-induced Mood Disorder, Alcohol-Induced Anxiety Disorder, Alcohol-induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-induced Anxiety Disorder, Amphetamine-induced Sexual Dysfunction, Amphetamine-induced Sleep Disorder and Amphetamine-Related Disorder Not Otherwise Specified (292.9); Caffeine Related Disorders such as Caffeine Intoxication (305.90), Caffeine-induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-induced Mood Disorder, Cocaine-induced Anxiety Disorder, Cocaine-Induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-induced Persisting Dementia, Inhalant-induced Psychotic Disorder, Inhalant-induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-induced Sleep Disorder including the subtypes Insomnia Type, Hypersomnia Type, Parasomnia Type and Mixed Type.

The compounds of formula (I) may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) may also be of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of formula (I) may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of formula (I) may also be of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt or solvate thereof. Treatment of epilepsy may be carried out by the administration of a non-toxic anticonvulsant effective amount of a compound of the formula (I) or a salt or solvate thereof.

The compounds of formula (I) may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

Other disorders include benign forgetfulness, childhood learning disorders and closed head injury, Parkinson's disease, dyskinetic disorders, cognitive impairment, emesis, movement disorders, amnesia, circadian rhythm disorders, aggression and vertigo.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

In another aspect of the invention, there is provided use of a compound of formula (I) as hereinbefore defined or a salt or solvate thereof in the preparation of a medicament for the treatment of a disorder mediated by GlyT1.

In one embodiment, the disorder mediated by GlyT1 to be treated by the use or method as hereinbefore described is a psychosis (including schizophrenia), dementia or an attention deficit disorder. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

The compounds of formula (I) and their salts and solvates thereof may also be suitable for combination with other active ingredients, such as typical and atypical antipsychotics, to provide improved treatment of psychotic disorders.

Thus, the present invention also provides:
i) a combination product comprising a compound of the invention and an anti psychotic;
ii) a pharmaceutical composition comprising a combination product as defined in i) above and at least one carrier, diluent or excipient;
iii) the use of a combination product as defined in i) above in the manufacture of a medicament for treating a disease or condition caused by a reduction or imbalance in glutamate receptor function in a mammal;
iv) a combination product as defined in i) above for use in treating a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination product as defined in i) above for use as a medicament;
vii) a method of treatment of a disease or condition caused by a reduction or imbalance in glutamate receptor function in a mammal comprising administering an effective amount of a combination product as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt or solvate thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt or solvate thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt or solvate thereof to a patient receiving therapeutic administration of at least one antipsychotic agent. In a further aspect, the invention provides the use of compounds of formula (I) or a salt or solvate thereof in the manufacture of a medicament for adjunctive therapeutic administration for the treatment of a psychotic disorder in a patient receiving therapeutic administration of at least one antipsychotic agent. The invention further provides compounds of formula (I) or a salt or solvate thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt or solvate thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt or solvate thereof. The invention further provides at least one antipsychotic agent for adjunctive therapeutic administration for the treatment of a psychotic disorder in a patient receiving therapeutic administration of compounds of formula (I) or a salt or solvate thereof.

In a further aspect, the invention provides a method of treatment of a psychotic disorder by simultaneous therapeutic administration of compounds of formula (I) or a salt or solvate thereof in combination with at least one antipsychotic agent. The invention further provides the use of a combination of compounds of formula (I) or a salt or solvate thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt or solvate thereof and at least one mood stabilising or antimanic agent, a pharmaceutical composition comprising compounds of formula (I) or a salt or solvate thereof and at least one mood stabilising or antimanic agent, the use of a pharmaceutical composition comprising compounds of formula (I) or a salt or solvate thereof and at least one mood stabilising or antimanic agent in the manufacture of a medicament for the treatment of a psychotic disorder, and a pharmaceutical composition comprising compounds of formula (I) or a salt or solvate thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Formulations suitable for rectal administration may be provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

The invention is further illustrated by the following non-limiting examples.

Where reactions are described as having been carried out in a similar manner to earlier, more completely described, reactions, the general reaction conditions used were essentially the same. Work up conditions used were of the types standard in the art, but may have been adapted from one reaction to another.

### Abbreviations:

THF tetrahydrofuran
DCM dichloromethane
DMF dimethylformamide
DMSO dimethylsulfoxide
BACH-EI borane-N-ethyl-N-isopropylaniline complex
BOC Benzyloxycarbonyl
HATU Q-(7-azabenzotriazol-1-yl)-N,N,N′,N′-tetramethyluroniumhexa fluorophosphate
EDC N-(3-(dimethylamino)propyl)-N-ethylcarbodiimide hydrochloride
HOAt 3H-(1,2,3)-triazolo(4,5-b)pyridine-3-ol
NMP N-methylpyrrolidinone
DIPEA N,N-diisopropylethylamine
HOBt 1-hydroxybenzotriazole hydrate
rt room temperature
0.88 ammonia concentrated aqueous ammonia specific gravity 0.88

### Analytical LC/MS Chromatography Conditions

### Conditions A:

Column: Waters Atlantis 50 mm×4.6 mm, 3 um particle size

Mobile phase:
A: 0.05% Formic acid+Water
B: Acetonitrile+0.05% Formic acid

Gradient: 5-min runtime: 3% B to 97% B over 4 min

Flow rate: 3 ml/min

UV wavelength range: 220-330 nm

Temperature: 30° C.

### Conditions B*:

Column: Phenomenex C₁₈(2) Luna, 30 mm×4.6 mm

Mobile phase:
A: Water
B: Acetonitrile

Gradient: 3.5 min runtime: 20% B to 100% B over 2.5 min

Flow rate: 2 ml/min

UV wavelength range: 214-254 nm

Temperature: 40° C.

### Conditions C**:

Column: Waters Atlantis 20 mm×4.6 mm, 3 um particle size

Mobile phase:
A: 0.1% Formic acid+Water
B: Acetonitrile+0.1% Formic acid

Gradient: 5.5-min runtime: 3% B to 97% B over 5.3 min

Flow rate: 1 ml/min

UV wavelength range: 210-350 nm

Temperature: Ambient

Conditions B were used for the analyses indicated with * in the Descriptions and Examples below and conditions C were used for the analyses indicated with ** in the Descriptions and Examples below. Conditions A were used for all other analyses.

### Description 1: [(1S)-1-Phenyl-2-(1-piperidinyl)ethyl]amine

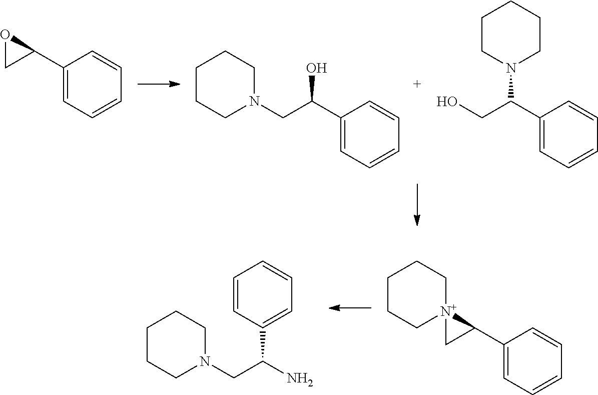

(S)-Styrene oxide (0.50 mL, 4.4 mmol, 1 eq) was added to a stirred solution of piperidine (0.69 mL, 7.0 mmol, 1.6 eq) in ethanol (15 mL) and the resulting mixture heated at reflux for 3 h. The solvent was evaporated at reduced pressure and the crude material thoroughly dried for 2 h under high vacuum to yield a crystalline product.

The crude product was redissolved in diethyl ether (20 mL) and triethylamine (1.8 mL, 13.2 mmol, 3 eq) was added. The reaction mixture was cooled to 0° C. under a nitrogen atmosphere and methanesulfonyl chloride (0.4 mL, 5.3 mmol, 1.2 eq) was added dropwise. The reaction mixture was stirred for 30 min at 0° C. during which time a white precipitate formed which made stirring difficult. Triethylamine (1.2 mL, 8.8 mmol, 2 eq) was added and the reaction mixture allowed to warm to rt. Aqueous 0.88 ammonia (11 mL, 650 mmol, 50 eq) was added and the resulting biphasic mixture stirred for 16 h at rt under an atmosphere of nitrogen.

The aqueous layer was separated and extracted three times with diethyl ether. The combined organics were washed with saturated. aqueous NaHCO₃ solution and water dried (Na₂SO₄) and concentrated at reduced pressure to give the crude product as a yellow oil.

The product was purified by Kugelrohr distillation (200-210° C., 0.2 mm Hg) to yield the title compound (0.58 g, 64%) as a light yellow oil. Mass Spectrum (Electrospray LC/MS): Found 205 (MH⁺). C₁₃H₂₀N₂ requires 204. Ret. Time 2.53 min.*

### Description 2: [(1S)-1-Phenyl-2-(1-pyrrolidinyl)ethyl]amine

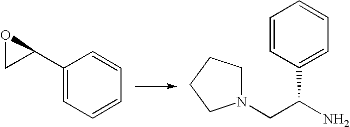

The title compound (1.50 g; 81%) was prepared from (S)-styrene oxide and pyrrolidine in a similar manner to that described in D1. ¹H NMR (CDCl₃) δ_{H}, 1.80 (6H, br s), 2.38 (2H, d), 2.49 (2H, m), 2.66 (2H, m), 2.80 (1H, t), 4.10 (1H, m), 7.2-7.42 (5H, m).

### Description 3: [(1S)-2-(2-Methyl-1-pyrrolidinyl)-1-phenylethyl]amine

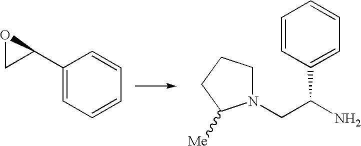

The title compound (0.54 g; 50%) was prepared from (S)-styrene oxide and 2-methylpyrrolidine in a similar manner to that described in D1. Mass Spectrum (Electrospray LC/MS): Found 205 (MH⁺). C₁₃H₂₀N₂ requires 204. Ret. Time 0.36 min.*

### Description 4: [(1S)-2-(2-Ethyl-1-pyrrolidinyl)-1-phenylethyl]amine

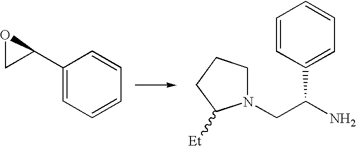

The title compound (0.133 g; 60%) was prepared from (S)-styrene oxide and 2-ethylpyrrolidine in a similar manner to that described in D1, except that the product was purified by silica gel column chromatography, eluting with dichloromethane-methanol-aqueous ammonia mixtures. Mass Spectrum (Electrospray LC/MS): Found 219 (MH⁺). C₁₄H₂₂N₂ requires 218. Ret. Time 0.39 min.

### Description 5: [(1S)-2-(2,4-Dimethyl-1-pyrrolidinyl)-1-phenylethyl]amine

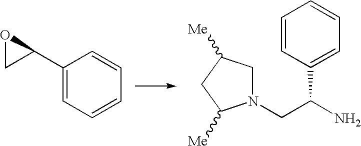

The title compound (0.100 g; 50%) was prepared from (S)-styrene oxide and 2,4-dimethylpyrrolidine in a similar manner to that described in D1, except that the product was purified by silica gel column chromatography, eluting with dichloromethane-methanol-aqueous ammonia mixtures. Mass Spectrum (Electrospray LC/MS): Found 219 (MH⁺). C₁₄H₂₂N₂ requires 218. Ret. Time 0.37 min.

### Description 6: [(2S)-2-Amino-2-phenylethyl]methyl(1-methylethyl)amine

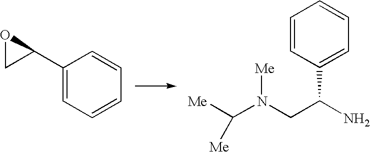

The title compound (0.514 g; 61%) was prepared from (S)-styrene oxide and isopropylmethylamine in a similar manner to that described in D1.

### Description 7: Methyl (2S)-({[2-chloro-3-(trifluoromethyl)phenyl]carbonyl}amino) (phenyl)ethanoate

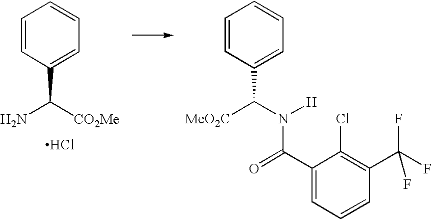

(S)-2-Phenylglycine methyl ester hydrochloride (0.201 g; 1.0 mmol) and 2-chloro-3-trifluoromethylbenzoyl chloride (0.243 g, 1 mmol)(prepared from 2-chloro-3-trifluoromethylbenzoic acid and thionyl chloride by the procedure described in Example 1) were stirred in dichloromethane (5 ml) in the presence of triethylamine (0.4 ml; 3 mmol) at room temperature under an atmosphere of argon for 16 hours. The organic solution was then washed with saturated sodium hydrogen carbonate solution, dried (MgSO₄) and evaporated at reduced pressure. The residue was chromatographed over silica gel (20 g), eluting with ethyl acetate/pentane mixtures to yield the title compound as a white solid (0.330 g; 89% yield). Mass Spectrum (Electrospray LC/MS): Found 372 (MH⁺). C₁₇H₁₃³⁵ClF₃NO₃ requires 371. Ret. Time 2.99 min.

### Description 8: 2-Chloro-N-[(1S)-2-oxo-1-phenylethyl]-3-(trifluoromethyl)benzamide

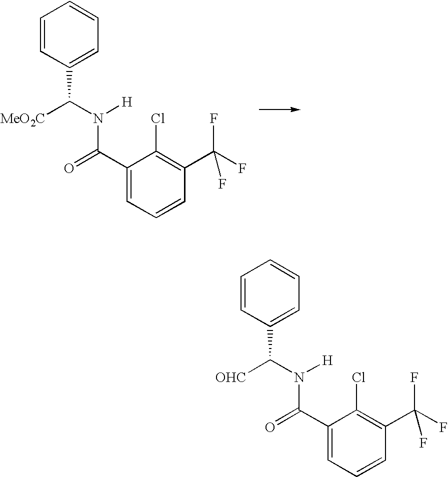

A solution of methyl (2S)-({[2-chloro-3-(trifluoromethyl)phenyl]carbonyl}amino) (phenyl)ethanoate (Description 7) (0.050 g; 0.135 mmol) in dichloromethane (2 ml) was cooled to −78° C. under an atmosphere of argon. A solution of diisobutyl aluminum hydride in toluene (0.266 ml of 1.5M solution; 0.18 mmol) was added and the resulting solution was stirred at −78° C. for 3 hours. Methanol (0.5 ml) was then added and the reaction solution allowed to warm up to room temperature. The reaction solution was diluted with dichloromethane, washed with brine, dried (MgSO₄) and evaporated at reduced pressure to yield the title compound (0.037 g) as a yellow gum.

### Description 9: (S)-(1-Phenyl-2-pyrrolidin-1-yl-ethyl)-carbamic acid tert-butyl ester and (S)-2-phenyl-aziridine-1-carboxylic acid tert-butyl ester

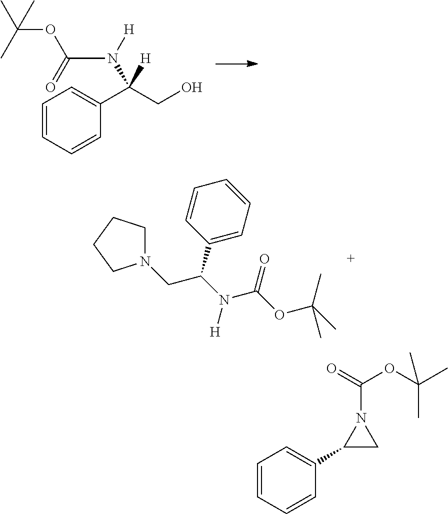

(S)—N-BOC-2-phenylglycinol (1 g, 4.2 mmol, 1 eq) was dissolved in acetonitrile (10 mL) and methanesulfonyl chloride (0.34 mL, 4.4 mmol, 1.05 eq) and triethylamine (0.88 mL, 6.3 mmol, 1.5 eq) were added. The reaction mixture was stirred for 1 h at rt under nitrogen. Further triethylamine (0.88 mL, 6.3 mmol, 1.5 eq) was added followed by pyrrolidine (0.33 g, 4.6 mmol, 1.1 eq) and the reaction mixture heated at reflux for 3 h. The solvent was removed at reduced pressure and the residue redissolved in dichloromethane (20 mL). The organics were washed with aqueous sodium hydroxide solution (1.0 M; 20 mL), water (10 mL) and brine (10 mL), dried (MgSO₄) and concentrated at reduced pressure to give the crude product as a yellow oil. The products were purified by flash column chromatography to yield a 5:2 mixture of the amine and the aziridine respectively (265 mg).

### Description 10: (2S)-2-(4-fluorophenyl)oxirane

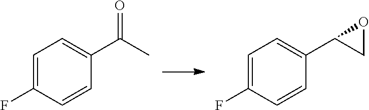

A stirred solution of 4-fluoroacetophenone (1.0 g, 7.2 mmol) in acetonitrile (15 ml) was treated with [hydroxy(tosyloxy)iodo]benzene (1.96 g, 5.0 mmol) and heated to reflux for 18 hours. The solvent was removed under reduced pressure and the residue chromatographed on silica gel (50 g). Elution with 0-30% ethyl acetate in pentane gave 2-(4-fluorophenyl)-2-oxoethyl 4-methylbenzenesulfonate as a white solid (1.2 g, 79%). δ_{H}: (400 MHz, CDCl₃) 2.45 (3H, s), 5.21 (2H, s), 7.15 (2H, m), 7.35 (2H, d, J=8.8 Hz), 7.84 (2H, d, J=8.8 Hz), 7.89 (2H, m) ppm. LC/MS: m/z (ES+) 309 (MH⁺, C₁₅H₁₃O₄SF requires 308), Retention time 3.09 minutes.

A solution of 2M BACH-El in tetrahydrofuran (2.0 ml, 4.0 mmol) and 1M (S)-(−)-2-methyl-CBS-oxazaborolidine in toluene (0.40 ml, 0.40 mmol) in dry THF (5 ml) was stirred under argon at room temperature and treated with a solution of 2-(4-fluorophenyl)-2-oxoethyl 4-methylbenzenesulfonate (1.2 g, 3.9 mmol) in dry THF (2 ml) added over 1 hour. After a further 30 minutes, water was added dropwise under argon until reaction ceased and the solvent was removed under reduced pressure. The residue was dissolved in ethyl acetate, washed twice with 2M hydrochloric acid, dried and evaporated to afford (2S)-2-(4-fluorophenyl)-2-hydroxyethyl 4-methylbenzenesulfonate as a colourless gum (1.14 g, quantitative). This was dissolved in diethyl ether (20 ml), treated with 2M sodium hydroxide solution (3 ml), and stirred overnight. The organic layer was dried and the solvent carefully removed under reduced pressure to give (2S)-2-(4-fluorophenyl)oxirane as an oil (470 mg, 68%). δ_{H}: (400 MHz, CDCl₃) 2.77 (1H, dd, J=5.4 and 2.6 Hz), 3.14 (1H, dd, J=5.4 and 4.0 Hz), 3.85 (1H, dd, J=4.0 and 2.6 Hz), 7.04 (2H, m), 7.25 (2H, m) ppm.

### Description 11: (1S)-1-(4-fluorophenyl)-2-(2-methyl-1-pyrrolidinyl)ethanamine

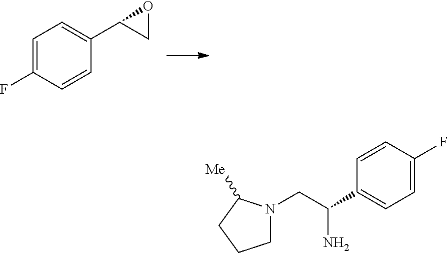

A solution of (2S)-2-(4-fluorophenyl)oxirane (100 mg, 0.72 mmol) (D9) and 2-methylpyrrolidine (147 ul, 1.44 mmol) in ethanol (3 ml) was heated to reflux for 18 hours. The solvent was removed under reduced pressure and the residue chromatographed on silica gel. Elution with ethyl acetate afforded (1S)-1-(4-fluorophenyl)-2-(2-methyl-1-pyrrolidinyl)ethanol as a colourless oil (116 mg, 68%).

A solution of (1S)-1-(4-fluorophenyl)-2-(2-methyl-1-pyrrolidinyl)ethanol (116 mg, 0.49 mmol) in DCM (3 ml) was cooled to 0° and treated with methanesulphonyl chloride (45 ul, 0.59 mmol) and triethylamine (140 ul, 1.0 mmol). Stirred at room temperature for 2 hours, washed with saturated sodium hydrogen carbonate, dried and evaporated to give a crude product which was dissolved in THF (2 ml) and treated with specific gravity 0.88 aqueous ammonia solution (0.5 ml). After stirring overnight at room temperature, the THF was removed under reduced pressure and the residue dissolved in DCM. This was washed with saturated sodium hydrogen carbonate, dried and evaporated to afford crude (1S)-1-(4-fluorophenyl)-2-(2-methyl-1-pyrrolidinyl)ethanamine. Used without further purification.

### Description 12: (1S)-1-(2-fluorophenyl)-2-(2-methyl-1-pyrrolidinyl)ethanamine

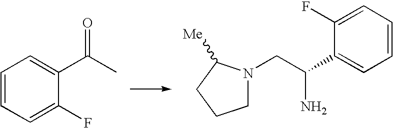

Prepared in similar manner as in D10 and D1.

### Description 13: (1S)-1-(3-fluorophenyl)-2-(2-methyl-1-pyrrolidinyl)ethanamine

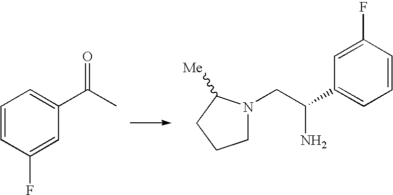

Prepared in similar manner as in D10 and D1.

### Description 14: (1S)-1-[2-(methyloxy)phenyl]-2-(2-methyl-1-pyrrolidinyl)ethanamine

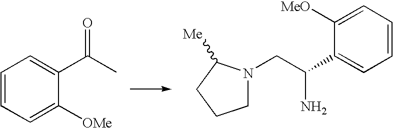

Prepared in similar manner as in D10 and D1.

### Description 15: (1S)-1-[3-(methyloxy)phenyl]-2-(2-methyl-1-pyrrolidinyl)ethanamine

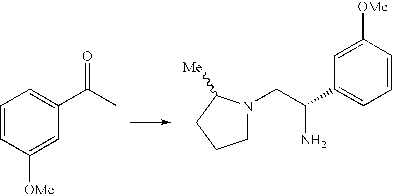

Prepared in similar manner as in D10 and D1.

### Description 16: (1S)-1-[4-(methyloxy)phenyl]-2-(2-methyl-1-pyrrolidinyl)ethanamine

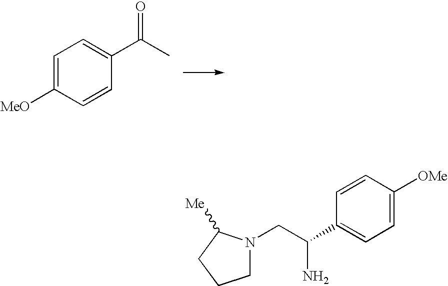

Prepared in similar manner as in D10 and D11.

### Description 17: 3-[(1S)-1-amino-2-(2-methyl-1-pyrrolidinyl)ethyl]benzonitrile

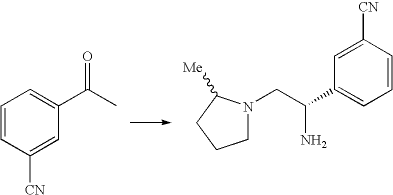

Prepared in similar manner as in D10 and D11.

### Description 18: 4-[(1S)-1-amino-2-(2-methyl-1-pyrrolidinyl)ethyl]benzonitrile

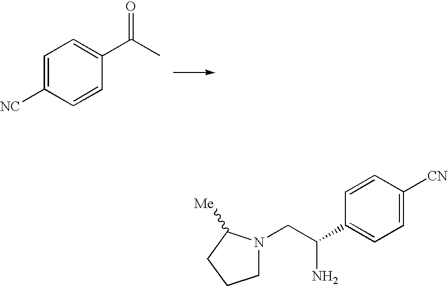

Prepared in similar manner as in D10 and D11.

### EXAMPLE 1

### 2-Chloro-N-[(1S)-1-phenyl-2-(1-piperidinyl)ethyl]-3-(trifluoromethyl)benzamide

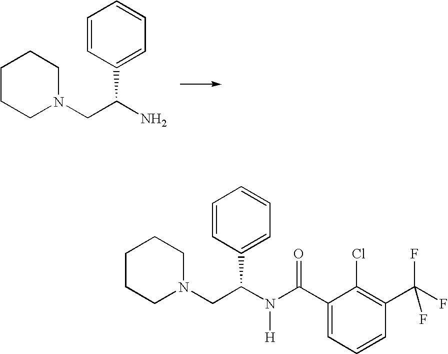

2-Chloro-3-trifluoromethyl-benzoic acid (112 mg, 0.5 mmol, 1 eq) was suspended in thionyl chloride (2 mL) and the mixture heated at reflux under an atmosphere of nitrogen for 1.5 h. The reaction mixture was concentrated at reduced pressure and the residue redissolved in dichloromethane (1 mL). The solution of the acyl chloride was added dropwise to a stirred solution of [(1S)-1-phenyl-2-(1-piperidinyl)ethyl]amine (102 mg, 0.5 mmol, 1 eq) and triethylamine (0.10 mL, 0.75 mmol, 1.5 eq) in dichloromethane (2 mL) at 0° C. under a nitrogen atmosphere. The reaction mixture was allowed to warm to rt and was stirred for 16 h. The reaction mixture was poured into aqueous citric acid (10%; 20 mL) and the aqueous then extracted with dichloromethane (3×20 mL). The combined organics were washed with saturated aqueous sodium hydrogen carbonate (30 mL), water (30 mL) and brine (30 mL), dried (MgSO₄) and concentrated at reduced pressure to give the crude product as an orange gum.

The crude product was recrystallised from diisopropyl ether to give the title compound as an off white solid (100 mg, 49%). ¹H NMR (DMSO) δ_{H}, 1.40-1.65 (6H, m), 2.30-2.80 (6H, m), 5.25 (1H, m), 7.28-7.50 (4H, m), 7.70 (2H, s), 7.99 (1H, d), 9.09 (1H, d). Mass Spectrum (Electrospray LC/MS): Found 411 (MH⁺). C₂₁H₂₂³⁵ClF₃N₂O requires 410. Ret. Time 4.07 min.*

### EXAMPLE 2

### 2-Chloro-N-[(1S)-1-phenyl-2-(1-pyrrolidinyl)ethyl]-3-(trifluoromethyl)benzamide

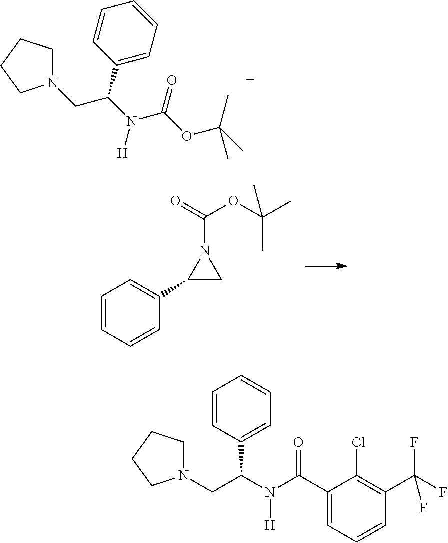

The reaction mixture from Description 9 (265 mg) was dissolved in dichloromethane (2 mL) and trifluoroacetic acid (2 mL) was added followed by 2 drops of water. The reaction mixture was stirred for 4 h and then toluene (5 mL) was added. The reaction mixture was concentrated at reduced pressure and the residue redissolved in dichloromethane (5 mL) and washed with saturated aqueous sodium hydrogen carbonate solution (5 mL) and water (5 mL). The organics were dried (MgSO₄) and concentrated at reduced pressure to give the crude amine as a yellow gum.

This amine was then amidated via the procedure described in Example 1. The product was purified by flash column chromatography, eluting with dichloromethane/methanol/aqueous ammonia mixtures, followed by triturating with diisopropyl ether/petroleum ether 40-60 to yield the title compound (13 mg). Mass Spectrum (Electrospray LC/MS): Found 397 (MH⁺). C₂₀H₂₀³⁵ClF₃N₂O requires 396. Ret. Time 3.92 min.*

### EXAMPLE 3

### 2-Chloro-N-[(1S)-2-(2-methyl-1-pyrrolidinyl)-1-phenylethyl]-3-(trifluoromethyl)benzamide

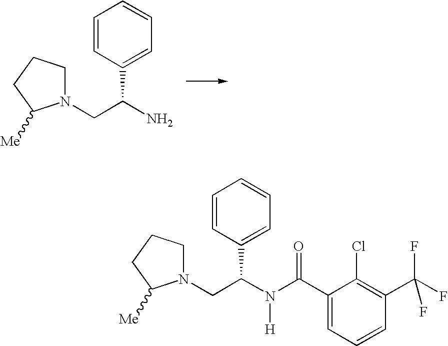

The title compound (0.102 g; 42%) was prepared from [(1S)-2-(2-methyl-1-pyrrolidinyl)-1-phenylethyl]amine and 2-chloro-3-trifluoromethyl-benzoic acid in a similar manner to that described in Example 1. ¹H NMR (CDCl₃) δ_{H}, 1.01 (3H, s), 1.30-3.35 (9H, m), 4.99 (0.5H, br s), 5.28 (0.5H, br s), 7.1-7.5 (7H, m), 7.79 (2H, m). Mass Spectrum (Electrospray LC/MS): Found 411 (MH⁺). C₂₁H₂₂³⁵ClF₃N₂O requires 410. Ret. Time 3.98 and 4.11 min (ratio of isomers 1:1).*

### EXAMPLE 4

### 2-Chloro-N-[(1S)-2-(2-ethyl-1-pyrrolidinyl)-1-phenylethyl]-3-(trifluoromethyl)benzamide

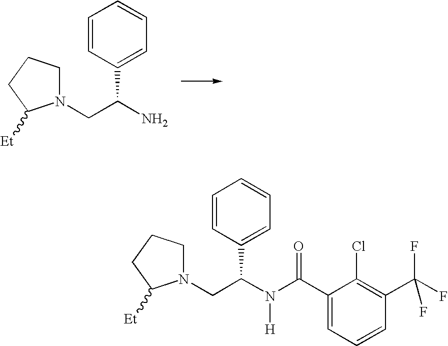

The title compound was prepared from [(1S)-2-(2-ethyl-1-pyrrolidinyl)-1-phenylethyl]amine and 2-chloro-3-trifluoromethyl-benzoic acid in a similar manner to that described in Example 1. ¹H NMR (MeOD) δ_{H}, 1.08 (3H, t), 1.51-1.62 (1H, m), 1.71-1.82 (1H, m), 1.99-2.12 (2H, m), 2.13-2.26 (1H, m), 2.30-2.40 (1H, m), 3.37-3.53 (2H, m), 3.59-3.68 (1H, m), 3.80-3.92 (2H, m), 5.53 (1H, t), 7.39-7.70 (7H, m), 7.90 (1H, d). Mass Spectrum (Electrospray LC/MS): Found 425 (MH⁺). C₂₂H₂₄³⁵ClF₃N₂O requires 424. Ret. Time 2.11 min.

### EXAMPLE 5

### 2-Chloro-N-[(1S)-2-(2,4-dimethyl-1-pyrrolidinyl)-1-phenylethyl]-3-(trifluoromethyl)benzamide

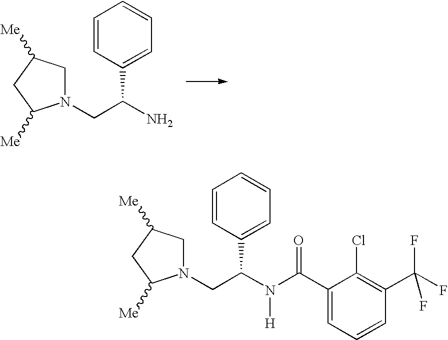

The title compound was prepared from [(1S)-2-(2,4-dimethyl-1-pyrrolidinyl)-1-phenylethyl]amine and 2-chloro-3-trifluoromethyl-benzoic acid in a similar manner to that described in Example 1. ¹H NMR (CDCl₃) δ_{H}, 1.18 (3H, 2×d), 1.40-1.58 (4H, m), 2.38-2.48 (1H, m), 2.52-2.70 (1H, m), 3.50-3.73 (3H, m), 3.80-3.98 (1H, m), 5.52 (1H, m), 7.39-7.90 (8H, m). Mass Spectrum (Electrospray LC/MS): Found 425 (MH⁺). C₂₂H₂₄³⁵ClF₃N₂O requires 424. Ret. Time 2.14 min.

### EXAMPLE 6

### 2-Chloro-N-{(1S)-2-[methyl(1-methylethyl)amino]-1-phenylethyl}-3-(trifluoromethyl)benzamide

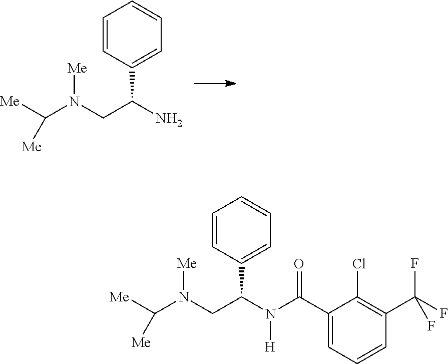

The title compound was prepared from [(2S)-2-amino-2-phenylethyl]methyl(1-methylethyl)amine and 2-chloro-3-trifluoromethyl-benzoic acid in a similar manner to that described in Example 1. Mass Spectrum (Electrospray LC/MS): Found 399 (MH⁺). C₂₀H₂₂³⁵ClF₃N₂O requires 398. Ret. Time 4.05 min.*

### EXAMPLE 7

### 2-Chloro-N-{(1S)-2-[(1-methylethyl)amino]-1-phenylethyl}-3-(trifluoromethyl)benzamide

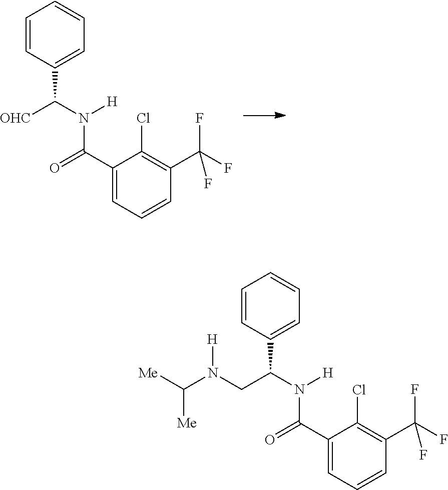

A mixture of 2-chloro-N-[(1S)-2-oxo-1-phenylethyl]-3-(trifluoromethyl)benzamide (Description 8) (0.037 g; 0.11 mmol), isopropylamine (0.009 ml; 0.11 mmol), sodium triacetoxyborohydride (0.025 g; 0.12 mmol) and acetic acid (0.007 ml; 0.12 mmol) in dichloromethane (2 ml) was stirred at room temperature under an atmosphere of argon for 16 hours. Saturated sodium hydrogen carbonate solution was then added dropwise and the organic layer was dried over MgSO₄. The solvent was evaporated at reduced pressure and the residue was chromatographed over silica gel (10 g), eluting with dichloromethane/methanol/ammonia mixtures, to yield the title compound as a white solid (0.003 g). ¹H NMR (CDCl₃) δ_{H}, 1.20 (6H, 2×d), 3.02 (1H, dd), 3.12 (1H, m), 3.49 (1H, m), 5.57 (1H, m), 7.32-7.45 (6H, m), 7.70-7.79 (2H, m), 8.30 (1H, br s), 9.89 (1H, br s). Mass Spectrum (Electrospray LC/MS): Found 385 (MH⁺). C₁₉H₂₀³⁵ClF₃N₂O requires 384. Ret. Time 2.07 min.

### EXAMPLE 8

### 2-chloro-N-[(1S)-1-(2-fluorophenyl)-2-(2-methyl-1-pyrrolidinyl)ethyl]-3-(trifluoromethyl)benzamide

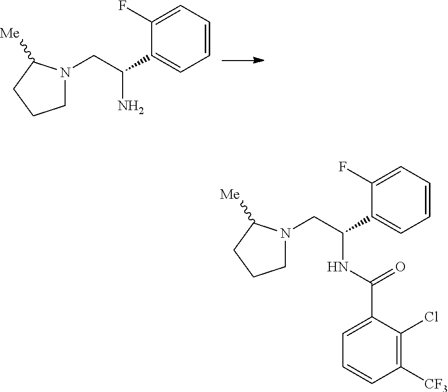

A solution of the crude amine (D12) (0.41 mmol) in DCM (3 ml) was treated with triethylamine (140 ul, 1.0 mmol) and 2-chloro-3-trifluoromethylbenzoyl chloride (99 mg, 0.41 mmol) and stirred at room temperature for 3 hours. The reaction mixture was washed with saturated sodium hydrogen carbonate, dried and evaporated to afford the crude product. Chromatography on silica gel (10 g) eluting with 20-100% ethyl acetate in pentane gave 2-chloro-N-[(1S)-1-(2-fluorophenyl)-2-(2-methyl-1-pyrrolidinyl)ethyl]-3-(trifluoromethyl)benzamide as a mixture of diastereoisomers (70 mg, 40%). δ_{H}: (400 MHz, CDCl₃) 1.0-1.05 (3H, d), 1.3-3.3 (9H, overlapping m), 5.29 and 5.45 (1H, m), 7.0-7.8 (8H, overlapping m) ppm. LC/MS: m/z (ES+) 429 (MH⁺, C₂₁H₂₁N₂OF4Cl requires 428), Retention time 2.23 minutes.

### EXAMPLE 9

### 2-chloro-N-[(1S)-1-(3-fluorophenyl)-2-(2-methyl-1-pyrrolidinyl)ethyl]-3-(trifluoromethyl)benzamide

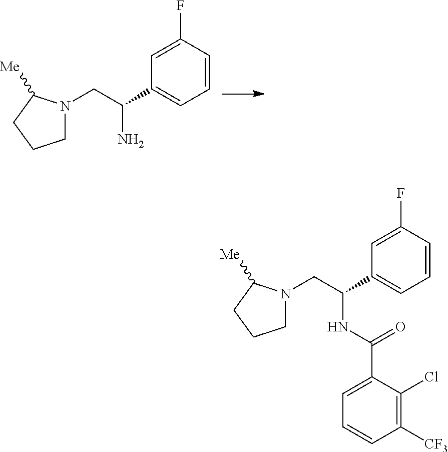

Prepared from D13 as a mixture of diastereoisomers in a similar manner to as outlined in Example 8. δ_{H}: (400 MHz, CDCl₃) 1.0-1.05 (3H, overlapping d), 1.3-3.3 (9H, overlapping m), 4.93 and 5.20 (1H, m), 7.0-7.8 (8H, overlapping m) ppm. LC/MS: m/z (ES+) 429 (MH⁺, C₂₁H₂₁N₂OF4Cl requires 428), Retention time 2.19 minutes.

### EXAMPLE 10

### 2-chloro-N-[(1S)-1-(4-fluorophenyl)-2-(2-methyl-1-pyrrolidinyl)ethyl]-3-(trifluoromethyl)benzamide

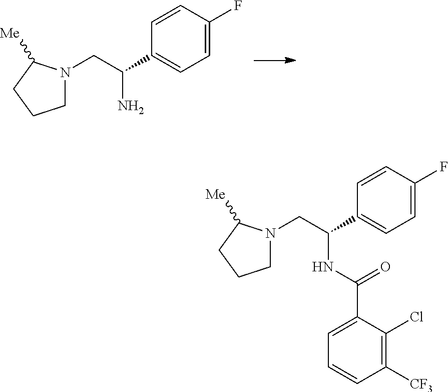

Prepared from D11 as a mixture of diastereoisomers in a similar manner to as outlined in Example 8. δ_{H}: (400 MHz, CDCl₃) 1.0-1.05 (3H, overlapping d), 1.3-3.3 (9H, overlapping m), 4.92 and 5.20 (1H, m), 7.0-7.8 (8H, overlapping m) ppm. LC/MS: m/z (ES+) 429 (MH⁺, C₂₁H₂₁N₂OF4Cl requires 428), Retention time 2.31 minutes.

### EXAMPLE 11

### 2-chloro-N-[(1S)-1-(2-methoxyphenyl)-2-(2-methyl-1-pyrrolidinyl)ethyl]-3-(trifluoromethyl)benzamide

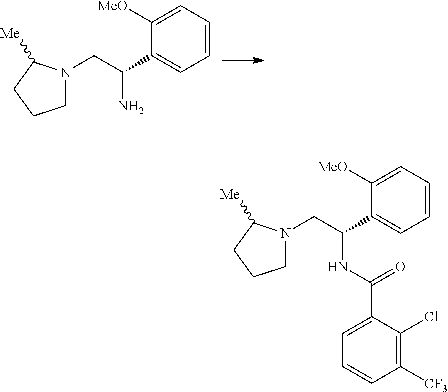

Prepared from D14 as a mixture of diastereoisomers in a similar manner to as outlined in Example 8. δ_{H}: (400 MHz, CDCl₃) 1.0 and 1.05 (3H, d), 1.3-3.3 (9H, overlapping m), 3.88 (3H, s), 5.41 and 5.47 (1H, overlapping m), 6.9-7.8 (8H, overlapping m) ppm. LC/MS: m/z (ES+) 441 (MH⁺, C₂₂H₂₄N₂O₂F₃Cl requires 440), Retention time 2.27 minutes.

### EXAMPLE 12

### 2-chloro-N-[(1S)-1-(3-methoxyphenyl)-2-(2-methyl-1-pyrrolidinyl)ethyl]-3-(trifluoromethyl)benzamide

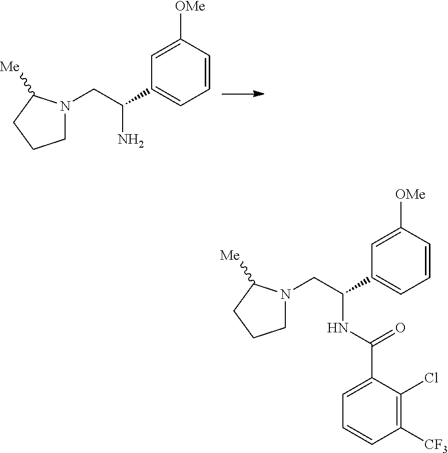

Prepared from D15 as a mixture of diastereoisomers in a similar manner to as outlined in Example 8. δ_{H}: (400 MHz, CDCl₃) 1.0-1.05 (3H, overlapping d), 1.3-3.25 (9H, overlapping m), 3.82 (3H, s), 4.95 and 5.20 (1H, m), 6.8-7.8 (8H, overlapping m) ppm. LC/MS: m/z (ES+) 441 (MH⁺, C₂₂H₂₄N₂O₂F₃Cl requires 440), Retention time 2.19 minutes.

### EXAMPLE 13

### 2-chloro-N-[(1S)-1-(4-methoxyphenyl)-2-(2-methyl-1-pyrrolidinyl)ethyl]-3-(trifluoromethyl)benzamide

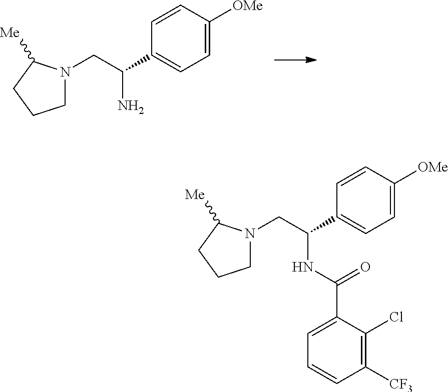

Prepared from D16 as a mixture of diastereoisomers in a similar manner to as outlined in Example 8. δ_{H}: (400 MHz, CDCl₃) 1.0-1.05 (3H, overlapping d), 1.3-3.25 (9H, overlapping m), 3.80 (3H, s), 4.93 and 5.20 (1H, m), 6.88 (2H, m), 7.1-7.8 (8H, overlapping m) ppm. LC/MS: m/z (ES+) 441 (MH⁺, C₂₂H₂₄N₂O₂F₃Cl requires 440), Retention time 2.19 minutes.

### EXAMPLE 14

### 2-chloro-N-[(1S)-1-(3-cyanophenyl)-2-(2-methyl-1-pyrrolidinyl)ethyl]-3-(trifluoromethyl)benzamide

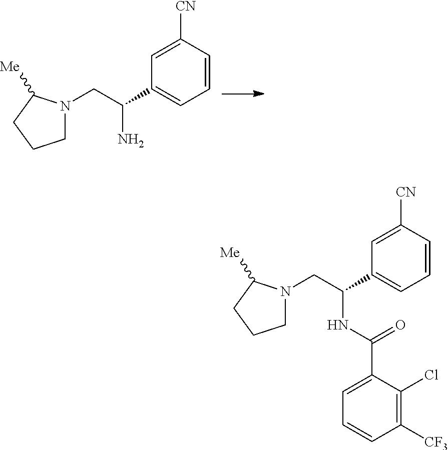

Prepared from D17 as a mixture of diastereoisomers in a similar manner to as outlined in Example 8. δ_{H}: (400 MHz, CDCl₃) 1.0-1.05 (3H, overlapping d), 1.3-3.25 (9H, overlapping m), 4.93 and 5.20 (1H, m), 7.3-7.8 (8H, overlapping m) ppm. LC/MS: m/z (ES+) 436 (MH⁺, C₂₂H₂₁N₃OF₃Cl requires 435), Retention time 2.15 minutes.

### EXAMPLE 15

### 2-chloro-N-[(1S)-1-(4-cyanophenyl)-2-(2-methyl-1-pyrrolidinyl)ethyl]-3-(trifluoromethyl)benzamide

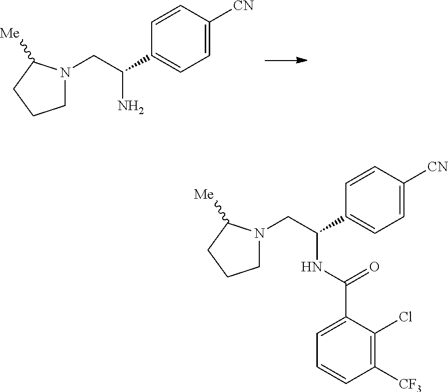

Prepared from D18 as a mixture of diastereoisomers in a similar manner to as outlined in Example 8. δ_{H}: (400 MHz, CDCl₃) 1.0-1.05 (3H, overlapping d), 1.3-3.25 (9H, overlapping m), 4.93 and 5.20 (1H, m), 7.3-7.8 (8H, overlapping m) ppm. LC/MS: m/z (ES+) 436 (MH⁺, C₂₂H₂₁N₃OF₃Cl requires 435), Retention time 2.0 minutes.

### EXAMPLE 16

### 4-Chloro-2-methyl-6-(methylthio)-N-[(1S)-1-phenyl-2-(1-pyrrolidinyl)ethyl]benzamide

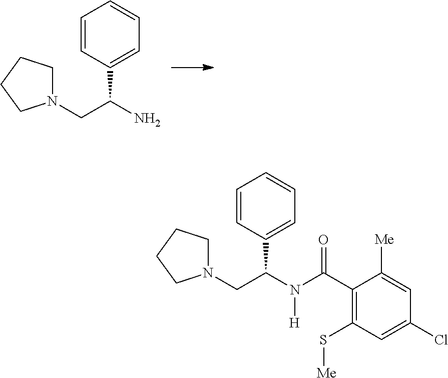

The compound of Example 16 was synthesised in a Robbins block as part of an array. The array method was as follows:

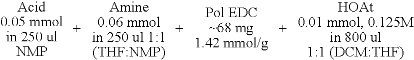

The acid used was 4-chloro-2-methyl-6-(methylthio)benzoic acid, obtained commercially. The amine used was the amine of description D2.

### Synthesis:

1. Pol-EDC (˜68 mg; 0.96 mmol) was added to the Robbins block using a 3.8 resin Loader.

2. 800 ul of 0.125 MHOAt solution was added to each well of the Robbins Block using a Hydra.

3. 250 ul of the acid solution in NMP was added to appropriate wells using Hydra

4. Finally 250 ul of the amine solution (THF:NMP;1:1) was added to the appropriate wells and the Robbins Block closed with the top sealing cover and allowed to shake for 60 h

5. Pol-Isocyanate resin (˜69 mg; 1.5 mmol/g loading) and Pol-CO3 (˜69 mg; 2.85 mmol/g loading) were added to scavenge the starting materials and allowed to mix over night. The products were passed through SCX blocks and the SCX blocks washed with DCM twice and MeOH twice. The products were then eluted from the SCX blocks with 0.5M NH3/MeOH into vials. Solvents were removed in a Genevac.

The yield was 10.1 mg, 26 umol. LCMS data: M+1; 389.16; retention time 2.43; purity 94%

The following Examples were made using methods similar to those disclosed herein, using analogous starting materials.

| Example | | |
|---|---|---|
| No. | Structure | Name |
| 17 | 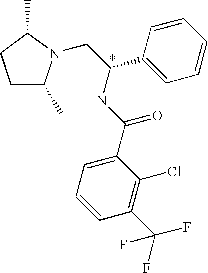 | 2-chloro-N-{(1S)-2-[(2R,5S)-2,5-dimethyl-1- pyrrolidinyl]-1-phenylethyl}-3- (trifluoromethyl)benzamide □□ |
| 18 | 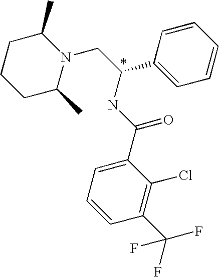 | 2-chloro-N-{(1S)-2-[(2R,6S)-2,6-dimethyl-1- piperidinyl]-1-phenylethyl}-3- (trifluoromethyl)benzamide □□ |
| 19 | 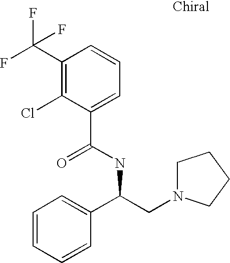 | 2-chloro-N-[(1 R)-1-phenyl-2-(1- pyrrolidinyl)ethyl]-3- (trifluoromethyl)benzamide □□ |
| 20 | 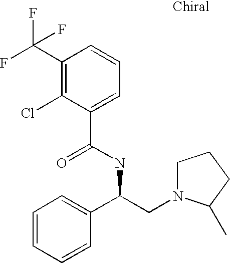 | 2-chloro-N-[(1R)-2-(2-methyl-1-pyrrolidinyl)- 1-phenylethyl]-3-(trifluoromethyl)benzamide □□ |
| 21 | 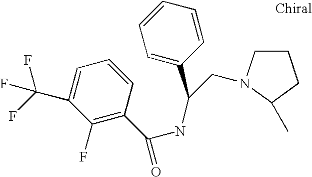 | 2-fluoro-N-[(1S)-2-(2-methyl-1-pyrrolidinyl)- 1-phenylethyl]-3-(trifluoromethyl)benzamide □□ |
| 22 | 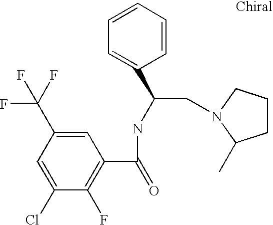 | 3-chloro-2-fluoro-N-[(1S)-2-(2-methyl-1- pyrrolidinyl)-1-phenylethyl]-5- (trifluoromethyl)benzamide □□ |
| 23 | 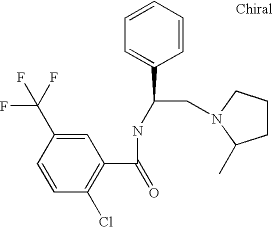 | 2-chloro-N-[(1S)-2-(2-methyl-1-pyrrolidinyl)- 1-phenylethyl]-5-(trifluoromethyl)benzamide □□ |
| 24 | 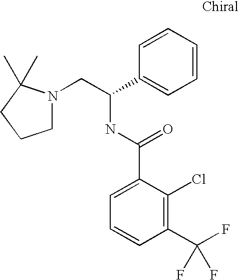 | 2-chloro-N-[(1S)-2-(2,2-dimethyl-1- pyrrolidinyl)-1-phenylethyl]-3- (trifluoromethyl)benzamide □□ |
| 25 | 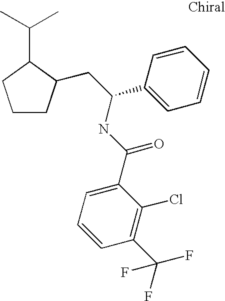 | 2-chloro-N-{(1S)-2-[2-(1-methylethyl)-1- pyrrolidinyl]-1-phenylethyl}-3- (trifluoromethyl)benzamide □□ |
| 26 | 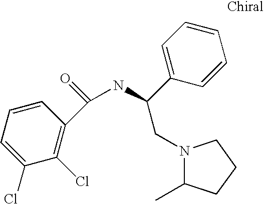 | 2,3-dichloro-N-[(1S)-2-(2-methyl-1- pyrrolidinyl)-1-phenylethyl]benzamide □□ |
| 27 | 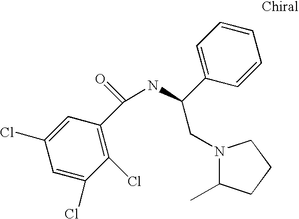 | 2,3,5-trichloro-N-[(1S)-2-(2-methyl-1- pyrrolidinyl)-1-phenylethyl]benzamide □□ □ |
| 28 | 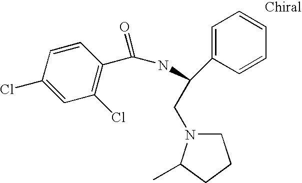 | 2,4-dichloro-N-[(1S)-2-(2-methyl-1- pyrrolidinyl)-1-phenylethyl]benzamide □□ |
| 29 | 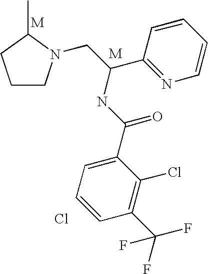 | 2-chloro-N-[2-(2-methyl-1-pyrrolidinyl)-1-(2- pyridinyl)ethyl]-3-(trifluoromethyl)benzamide □□ |
| 30 | 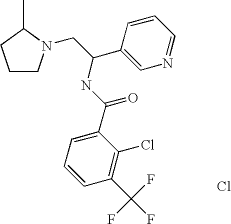 | 2-chloro-N-[2-(2-methyl-1-pyrrolidinyl)-1-(3- pyridinyl)ethyl]-3-(trifluoromethyl)benzamide □□ |
| 31 | 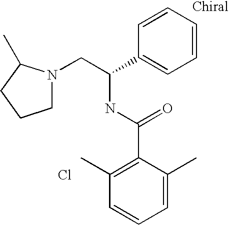 | 2,6-dimethyl-N-[(1S)-2-(2-methyl-1- pyrrolidinyl)-1-phenylethyl]benzamide |
| 32 | 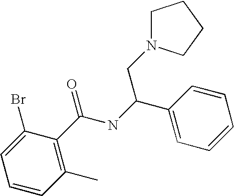 | 2-bromo-6-methyl-N-[1-phenyl-2-(1- pyrrolidinyl)ethyl]benzamide |

The compounds of the Examples above were converted or are convertable to their corresponding hydrochloride salts by dissolving the parent free base in DCM or DCM/methanol mixtures and adding 1M hydrogen chloride in ether, followed by evaporation and drying in vacuo.

1-15. (canceled)

16. A compound of formula (I) or a salt or solvate thereof: 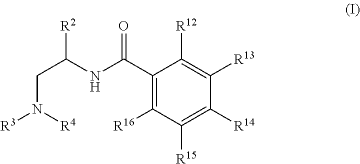
R² is phenyl unsubstituted or substituted with 1 or 2 R¹ groups, or pyridyl unsubstituted or substituted with 1 or 2 R¹ groups;
each R¹ is independently halo, C₁₋₄alkyl, C₁₋₄alkoxy, haloC₁₋₄alkyl, haloC₁₋₄alkoxy or cyano;
R³ is hydrogen or C₁₋₂ alkyl;
R⁴ is ethyl, n-propyl, i-propyl, n-butyl, i-butyl or t-butyl;
or R³ and R⁴ together with the nitrogen atom to which they are attached form a saturated 5- or 6-membered heterocyclic ring optionally substituted with one or more groups X;
each X is C₁₋₄alkyl or haloC₁₋₄alkyl;
R¹² is selected from the group consisting of hydrogen, fluoro, chloro, bromo, methyl and methylthio;
R¹³ is hydrogen, chloro or trifluoromethyl;
R¹⁴ is hydrogen, trifluoromethyl or chloro;
R¹⁵ is hydrogen, chloro or trifluoromethyl;
R¹⁶ is hydrogen, methyl, fluoro or chloro; and
Wherein R¹², R¹³, R¹⁴, R¹⁵ and R¹⁶ not all simultaneously being hydrogen.

17. A compound as claimed in claim 16 wherein R² is phenyl, unsubstituted or substituted with 1 or 2 R¹ groups, wherein each R¹ is independently halo, C₁₋₂alkyl, C₁₋₂alkoxy, haloC₁₋₂alkyl, haloC₁₋₂alkoxy or cyano.

18. A compound as claimed in claim 16 wherein R³ is hydrogen or methyl, and R⁴ is ethyl, n-propyl or i-propyl.

19. A compound as claimed in claim 16 wherein R³ and R⁴ together with the nitrogen atom to which they are attached form a saturated 5- or 6-membered heterocyclic ring optionally substituted with one or more groups X, wherein X is independently selected from the group consisting of C₁₋₂alkyl and haloC₁₋₂alkyl.

20. A compound as claimed in claim 16 wherein simultaneously: R¹² is chloro, R¹³ is trifluoromethyl, R¹⁴ is hydrogen, R¹⁵ is hydrogen, and R¹⁶ is hydrogen.

21. A compound as claimed in claim 16, which is
2-chloro-N-[(1S)-1-phenyl-2-(1-piperidinyl)ethyl]-3-(trifluoromethyl)benzamide
2-chloro-N-[(1S)-1-phenyl-2-(1-pyrrolidinyl)ethyl]-3-(trifluoromethyl)benzamide
2-chloro-N-[(1S)-2-(2-methyl-1-pyrrolidi nyl)-1-phenylethyl]-3-(trifluoromethyl)benzamide
2-chloro-N-[(1S)-2-(2-ethyl-1-pyrrolidi nyl)-1-phenylethyl]-3-(trifluoromethyl)benzamide
2-chloro-N-[(1S)-2-(2,4-dimethyl-1-pyrrolidi nyl)-1-phenylethyl]-3-(trifluoromethyl)benzamide
2-chloro-N-{(1S)-2-[methyl(1-methylethyl)amino]-1-phenylethyl}-3-(trifluoromethyl)benzamide
2-chloro-N-{(1S)-2-[(1-methylethyl)amino]-1-phenylethyl}-3-(trifluoromethyl)benzamide
2-chloro-N-[(1S)-1-(2-fluorophenyl)-2-(2-methyl-1-pyrrolidinyl)ethyl]-3-(trifluoromethyl)benzamide
2-chloro-N-[(1S)-1-(3-fluorophenyl)-2-(2-methyl-1-pyrrolidinyl)ethyl]-3-(trifluoromethyl)benzamide
2-chloro-N-[(1S)-1-(4-fluorophenyl)-2-(2-methyl-1-pyrrolidinyl)ethyl]-3-(trifluoromethyl)benzamide
2-chloro-N-[(1S)-1-(2-methoxyphenyl)-2-(2-methyl-1-pyrrolidinyl)ethyl]-3-(trifluoromethyl)benzamide
2-chloro-N-[(1S)-1-(3-methoxyphenyl)-2-(2-methyl-1-pyrrolidinyl)ethyl]-3-(trifluoromethyl)benzamide
2-chloro-N-[(1S)-1-(4-methoxyphenyl)-2-(2-methyl-1-pyrrolidinyl)ethyl]-3-(trifluoromethyl)benzamide
2-chloro-N-[(1S)-1-(3-cyanophenyl)-2-(2-methyl-1-pyrrolidinyl)ethyl]-3-(trifluoromethyl)benzamide
2-chloro-N-[(1S)-1-(4-cyanophenyl)-2-(2-methyl-1-pyrrolidinyl)ethyl]-3-(trifluoromethyl)benzamide
4-chloro-2-methyl-6-(methylthio)-N-[(1S)-1-phenyl-2-(1-pyrrolidinyl)ethyl]benzamide
2-chloro-N-{(1S)-2-[(2R,5S)-2,5-dimethyl-1-pyrrolidinyl]-1-phenylethyl}-3-(trifluoromethyl)benzamide
2-chloro-N-{(1S)-2-[(2R,6S)-2,6-dimethyl-1-piperidinyl]-1-phenylethyl}-3-(trifluoromethyl)benzamide
2-chloro-N-[(1R)-1-phenyl-2-(1-pyrrolidinyl)ethyl]-3-(trifluoromethyl)benzamide
2-chloro-N-[(1R)-2-(2-methyl-1-pyrrolidinyl)-1-phenylethyl]-3-(trifluoromethyl)benzamide
2-fluoro-N-[(1S)-2-(2-methyl-1-pyrrolidinyl)-1-phenylethyl]-3-(trifluoromethyl)benzamide
3-chloro-2-fluoro-N-[(1S)-2-(2-methyl-1-pyrrolidinyl)-1-phenylethyl]-5-(trifluoromethyl)benzamide
2-chloro-N-[(1S)-2-(2-methyl-1-pyrrolidinyl)-1-phenylethyl]-5-(trifluoromethyl)benzamide
2-chloro-N-[(1S)-2-(2,2-dimethyl-1-pyrrolidi nyl)-1-phenylethyl]-3-(trifluoromethyl)benzamide
2-chloro-N-{(1S)-2-[2-(1-methylethyl)-1-pyrrolidi nyl]-1-phenylethyl}-3-(trifluoromethyl)benzamide
2,3-dichloro-N-[(1S)-2-(2-methyl-1-pyrrolidinyl)-1-phenylethyl]benzamide
2,3,5-trichloro-N-[(1S)-2-(2-methyl-1-pyrrolidinyl)-1-phenylethyl]benzamide
2,4-dichloro-N-[(1S)-2-(2-methyl-1-pyrrolidinyl)-1-phenylethyl]benzamide
2-chloro-N-[2-(2-methyl-1-pyrrolidinyl)-1-(2-pyridinyl)ethyl]-3-(trifluoromethyl)benzamide
2-chloro-N-[2-(2-methyl-1-pyrrolidinyl)-1-(3-pyridinyl)ethyl]-3-(trifluoromethyl)benzamide
2,6-dimethyl-N-[(1S)-2-(2-methyl-1-pyrrolidinyl)-1-phenylethyl]benzamide
2-bromo-6-methyl-N-[1-phenyl-2-(1-pyrrolidi nyl)ethyl]benzamide or a salt or solvate thereof.

22. A method of treating a human suffering from schizophrenia, dementia or attention deficit disorder which comprises administering an effective amount of a compound of formula (I) according to claim 16 to a patient in need thereof.

23. A pharmaceutical composition comprising a compound of formula (I) according to claim 16 and at least one carrier, diluent or excipient.

24. A pharmaceutical composition as claimed in claim 23 further comprising one or more other therapeutic agents, selected from the group consisting of an antidepressant agents selected from the group consisting of 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists, anticonvulsant agents; atypical antipsychotic drugs and cognitive enhancers.

25. A method of preparing a compound of formula (I) according to claim 16, comprising the step of:
reacting a compound of formula (II): 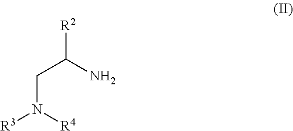
wherein
R² is phenyl unsubstituted or substituted with 1 or 2 R¹ groups, or pyridyl unsubstituted or substituted with 1 or 2 R¹ groups;
each R¹ is independently halo, C₁₋₄alkyl, C₁₋₄alkoxy, haloC₁₋₄alkyl, haloC₁₋₄alkoxy or cyano;
R³ is hydrogen or C₁₋₂ alkyl;
R⁴ is ethyl, n-propyl, i-propyl, n-butyl, i-butyl or t-butyl;
or R³ and R⁴ together with the nitrogen atom to which they are attached form a saturated 5- or 6-membered heterocyclic ring optionally substituted with one or more groups X;
with a compound of formula (III): 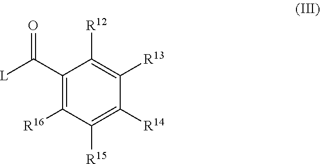
wherein
R¹² is selected from the group consisting of hydrogen, fluoro, chloro, bromo, methyl and methylthio;
R¹³ is hydrogen, chloro or trifluoromethyl;
R¹⁴ is hydrogen, trifluoromethyl or chloro;
R¹⁵ is hydrogen, chloro or trifluoromethyl;
R¹⁶ is hydrogen, methyl, fluoro or chloro; and
Wherein R¹², R¹³, R¹⁴, R¹⁵ and R¹⁶ not all simultaneously being hydrogen.
and L represents a suitable leaving group;
and thereafter optionally:
removing any protecting groups and/or
converting a compound of formula (I) into another compound of formula (I) and/or
forming a salt or solvate.

26. A compound of formula (II): 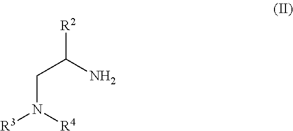
wherein R² is phenyl unsubstituted or substituted with 1 or 2 R¹ groups, or pyridyl unsubstituted or substituted with 1 or 2 R¹ groups;
each R¹ is independently halo, C₁₋₄alkyl, C₁₋₄alkoxy, haloC₁₋₄alkyl, haloC₁₋₄alkoxy or cyano;
R³ is hydrogen or C₁₋₂ alkyl;
R⁴ is ethyl, n-propyl, i-propyl, n-butyl, i-butyl or t-butyl;
or R³ and R⁴ together with the nitrogen atom to which they are attached form a saturated 5- or 6-membered heterocyclic ring optionally substituted with one or more groups X.
